(19) United States
(12) Patent Application Publication
(10) Pub. No.: US 2025/0341514 A1
(43) Pub. Date: Nov. 6, 2025

(54) COMPOSITIONS FOR USE WITH MULTIPLE FLUOROPHORES AND METHODS OF USING

(71) Applicant: BioLegend, Inc., San Diego, CA (US)

(72) Inventors: Tunde Mester, San Diego, CA (US); Anagha Ashok Divekar, San Diego, CA (US)

(21) Appl. No.: 19/184,045

(22) Filed: Apr. 21, 2025

Related U.S. Application Data
(60) Provisional application No. 63/640,919, filed on May 1, 2024.

(51) Int. Cl. G01N 33/53; G01N 33/533; G01N 33/68

(52) U.S. Cl. CPC G01N 33/5306; G01N 33/533; G01N 33/6872

(57) ABSTRACT

Improved compositions and methods of using the compositions in detecting the presence or absence of an analyte are disclosed. These compositions may provide improved signal with fewer false double positive populations observed, by dramatically reducing stacking of polymer dyes and allowing the use of multiple polymer dyes in a single tube thereby improving methods of using such compositions for fluorescent analyses.

### CROSS REFERENCE TO RELATED APPLICATIONS

This application depends from and claims priority to U.S. Provisional Application No. 63/640,919 filed May 1, 2024, the entire contents of which are incorporated herein by reference.

### FIELD

The present description relates to compounds for improving studies involving detection of a target analyte using fluorescent labeling, methods of making, and methods of using.

### BACKGROUND

Detecting, tracing and identifying biological materials are increasingly important in medical diagnoses, treatment, and research. The advancement of imaging technologies that are rapid, robust and are capable of detecting desired targets with low error are essential to these studies. Fluorescence technologies are increasingly utilized due to detection power and safety relative to other detection methodologies. The use of multiple fluorophores in a single solution for detection represents a powerful method for identification of multiple target types in a single sample.

Antibody cocktails are commonly custom made per the user's desired target collection. These cocktails commonly involve use of three or more antibodies and often over 20 different antibodies simultaneously. The presence of numerous antibodies in a single cocktail require individual dyes so as to be discernable from other antibodies in the system. Often the system requires the use of multiple polymer dyes as these dyes offer advantages over single dyes including the ability to tune fluorescent properties and improve signal. Polymer dyes, however, tend to stack against each other when mixed and stored together, creating false double positive populations observed during analysis.

Prior solutions to polymer dye stacking included keeping multiple polymer conjugated antibodies separated prior to use at the site of analysis. This is inconvenient for users at least as multiple vials need to be handled, and also slows down the overall process due to increased staining time and increasing chance of error. The problem of stacking of polymer dyes also prevents creation of dry down cocktails that are of increasing demand due to the ability to separate sample collection and analysis both temporally and physically.

Thus, there is a continuing need for improved compositions that can be used to improve analyses using multiple dyes in a single tube, and methods of using such compositions.

### SUMMARY

The following summary of the disclosure is provided to facilitate an understanding of some of the innovative features unique to the disclosure and is not intended to be a full description. A full appreciation of the various aspects of the disclosure can be gained by taking the entire specification, claims, drawings, and abstract as a whole.

Improved compositions and methods of using the compositions in detecting the presence or absence of an analyte are disclosed. These compositions may provide improved signal with fewer false double positive populations observed by dramatically reducing stacking of polymer dyes and allowing the use of multiple polymer dyes in a single tube thereby improving methods of using such compositions for fluorescent analyses.

As such, provided are buffering compositions that include one or more buffering agents, one or more celluloses or derivatives thereof, the celluloses or derivatives thereof at a concentration of less than about 2 wt % of the buffering composition; and polyvinyl alcohol at less than about 2 wt % of the buffering composition. A cellulose is optionally a linear or branched alkyl cellulose. An alkyl cellulose is optionally a C₁-C₄ alkyl cellulose. In some aspects, a cellulose is a methyl cellulose. Optionally a cellulose is a hydroxypropyl cellulose. Optionally, a cellulose is a branched chain alkyl cellulose. In some aspects, two or more celluloses are included, optionally the cellulose is a combination of methyl cellulose and hydroxypropyl cellulose. In some aspects, a cellulose is present at about 0.5 wt % to about 1 wt %, optionally about 0.75 wt % to about 1 wt %. In any of the aspects as provided herein, a buffering agent is optionally characterized by a pKa of 6-8. Optionally, a buffering agent includes a phosphate, optionally as phosphate buffered saline. Optionally, a buffering composition further includes a sugar, an albumin, or both. Optionally, an albumin is bovine serum albumin or human serum albumin. Optionally, an albumin is present at about 0.5 wt %. In some aspects, a buffering composition includes about 0.75 wt % to 1.5 wt % methyl cellulose and about 0.5 wt % polyvinyl alcohol, optionally about 1 wt % methyl cellulose or about 0.75 wt % to about 1 wt % hydroxypropyl cellulose, and about 0.5 wt % polyvinyl alcohol, optionally about 1 wt % methyl cellulose or about 0.75 wt % hydroxypropyl cellulose, and further comprising about 0.5 wt % polyvinyl alcohol.

Also provided are methods of analyzing a biological sample that may include obtaining a biological sample, forming an analysis composition by contacting the biological sample with a cocktail comprising the buffer composition as described in the foregoing paragraph or otherwise herein and one or more antibodies, the one or more antibodies conjugated to one or more florescent dyes. Optionally, the cocktail includes two or more antibodies specific for different target molecules, wherein an antibody specific for a first target molecule is conjugated to a first dye, and wherein an antibody specific for a second target molecule is conjugated to a second dye, the first dye and the second dye characterized by different fluorescent properties (e.g. absorption wavelength width, emission wavelength width, absorption maximum, emission maximum, Stokes shift, lifetime, anisotropy, photon yield, signal intensity, etc.). Optionally, the cocktail includes 3 to 23 antibodies directed to different target molecules and each conjugated to a different dye. In some aspects, at least two of the antibodies are conjugated to different brilliant violet dyes. Optionally, a dye is a polymer dye. A polymer dye is optionally Brilliant Violet 421™, Brilliant Violet 510™, Brilliant Violet 570™, Brilliant Violet 605™, Brilliant Violet 650™, Brilliant Violet 711™, Brilliant™ Violet 750, or Brilliant Violet 785™. Optionally, in place of or in addition to a polymer dye, a dye is optionally a rhodamine dye, a fluorescein dye, a coumarin dye, an indole dye, or any combination thereof. Optionally, a dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor®350, Alexa Fluor®430, Alexa Fluor®488, Alexa Fluor®532, Alexa Fluor®546, Alexa Fluor®568, Alexa Fluor®594, Alexa Fluor®633, Alexa Fluor®647, Alexa Fluor®660, Alexa Fluor®680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof. A method as provided herein optionally includes drying the analysis sample, wherein the cocktail includes methyl cellulose and polyvinyl alcohol, optionally about 1 wt % methyl cellulose and about 0.5 wt % polyvinyl alcohol. Optionally, in a method as provided herein includes use of a cocktail including methyl cellulose or hydroxypropyl cellulose, and further including polyvinyl alcohol, optionally about 1 wt % methyl cellulose, about 0.75 wt % to 1 wt % hydroxypropyl cellulose, and about 0.5 wt % polyvinyl alcohol. Optionally, a cocktail includes about 0.75 wt % methyl cellulose or about 0.75 wt % hydroxypropyl cellulose, and further including about 0.5 wt % polyvinyl alcohol. In any method as provided herein, the sample may be analyzed by flow cytometry, FISH, immunohistochemistry, sandwich assay, Southern blot, western blot, microarray, or substrate binding assay.

Also provided are methods of forming an analysis composition that may be used for analysis of a biological sample that include forming a cocktail including the buffering composition as described herein in any aspect and one or more antibodies, the one or more antibodies conjugated to one or more florescent dyes; and drying the analysis composition. Optionally, the cocktail includes methyl cellulose and polyvinyl alcohol, optionally about 1 wt % methyl cellulose and about 0.5 wt % polyvinyl alcohol. Optionally, the dyes include one or more polymer dyes, optionally Brilliant Violet 421™, Brilliant Violet 570™, Brilliant Violet 605™, Brilliant Violet 650™, Brilliant Violet 711™, or Brilliant Violet 785™. Optionally, in place of or in addition to a polymer dye, a dye may include a rhodamine dye, a fluorescein dye, a coumarin dye, an indole dye, or any combination thereof. Optionally, a method further includes reconstituting the analysis composition after the step of drying. Optionally, the reconstituting is by combining the analysis composition with a liquid biological sample.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings.

FIG. 1A illustrates studies using flow cytometry with polymer dye-conjugated antibodies revealing the presence of false double positive populations.

FIG. 1B illustrates that using the same antibodies as in FIG. 1A, the presence of a buffer composition as provided herein reduces the prevalence of false double positive populations.

FIG. 2 illustrates studies using flow cytometry with polymer dye-conjugated antibodies revealing the presence of false double positive populations that are absent when a buffer composition as provided herein is combined with the antibodies.

FIG. 3 illustrates studies using flow cytometry with polymer dye-conjugated antibodies revealing the presence of false double positive populations that are absent when two different buffer compositions as provided herein are combined with the antibodies.

FIG. 4 illustrates studies using flow cytometry with polymer dye-conjugated antibodies revealing the presence of false double positive populations that are absent when two different buffer compositions as provided herein are combined with the antibodies.

FIG. 5 illustrates studies using flow cytometry with polymer dye-conjugated antibodies revealing the presence of false double positive populations that are absent when antibodies are combined with a buffer composition as provided herein.

### DETAILED DESCRIPTION

Among the challenges remaining in florescent detection technologies, particularly in complex systems that employ polymer dyes, is the ability to combine multiple dyes in a single chamber in a way that provide stable interactions, but also prevents unwanted interactions with other antibodies or dye molecules in a system. Disclosed are unique solution systems that enable not only specific binding of a target molecule, but also prevent dye stacking such as occurs during dry-down procedures in complex antibody/dye cocktails. Thus, the disclosed compositions and methods improve detectability of target analytes in complex systems.

The term “complex” as used herein is intended to mean a single solution either before or after dry down if used that includes two or more antibody types wherein at least two different antibody types include differing dyes conjugated thereto.

When used to describe certain carbon atom-containing chemical groups, a parenthetical expression having the form “Cx-Cy” means that the unsubstituted form of the chemical group has from x carbon atoms to y carbon atoms, inclusive of x and y. For example, a C2-C4 alkyl is an alkyl group or radical having from 2 to 4 carbon atoms in its unsubstituted form. As another example, C1-C4 alkyl is an alkyl group or radical having from 1 to 4 carbon atoms in its unsubstituted form. A carbon atom-containing chemical group may be or include an alkyl, alkenyl, or alkynyl radical, and may be linear, branched or cyclic. Such carbon atom-containing chemical groups may be substituted or unsubstituted, and/or may include one or more substituent molecules in place of a hydrogen.

The term “alkyl” means a saturated straight or branched saturated hydrocarbon radical of from x to y carbon atoms. The carbons of the radical may be substituted at one or more hydrogens with a non-hydrogen molecule or group or unsubstituted.

The term “alkenyl” means an unsaturated straight or branched hydrocarbon radical of from x to v carbon atoms and includes at least one carbon-carbon double bond. The carbons of the radical may be substituted at one or more hydrogens with a non-hydrogen molecule or group or unsubstituted.

The terms “alkynyl” means an unsaturated straight or branched hydrocarbon radical of from x to y carbon atoms and includes at least one carbon-carbon triple bond. The carbons of the radical may be substituted or unsubstituted.

The term “cycloalkyl” means a saturated cyclic hydrocarbon radical of from x to y carbon atoms. For example, Cx-Cy cycloalkyl is defined as having from x to y carbon atoms. Examples of unsubstituted (C₃-C₅) cycloalkyl are cyclopropyl, cyclobutyl, cyclopentyl, cyclohexyl, cycloheptyl, cyclooctyl. A cyclic hydrocarbon radical may be or include an alkyl, alkenyl, or alkynyl radical. Such carbon atom-containing chemical groups may be substituted or unsubstituted, and/or may include one or more substituent molecules in place of a hydrogen.

The term “saturated” means lacking carbon-carbon double bonds, carbon-carbon triple bonds, and (in heteroatom-containing groups) carbon-nitrogen, carbon-phosphorous, carbo-oxygen or carbon-sulfur double bonds. The term “unsaturated” means containing one or more carbon-carbon double bonds, carbon-carbon triple bonds, and (in heteroatom-containing groups) carbon-nitrogen, carbon-phosphorous, carbon-oxygen, or carbon-sulfur double bonds.

The term “heteroatom,” refers to an atom other than hydrogen or carbon. Examples of groups containing one or more than one heteroatom include P(R^{P})₂, P(R^{P}), N(R^{N})₂, N(R^{N}), N, O, OR^{C}, S, SR^{C}, S(O), and S(O)₂, or other groups containing one or more of the foregoing wherein R is a linear, branched, or cyclic alkyl, alkenyl, or alkynl.

The term “heterocycle” means a cyclic, optionally fused cyclic, radical containing any number equal to or greater than 3, optionally 4-10, total ring atoms of which at least one atom is a carbon atom. The heteroatoms of the heterocycles may include N(R^{N})₂, N(R^{N}), N, O, OR^{C}, S, SR^{C}, S(O), and S(O)₂, wherein each of the heteroalkyl are unsubstituted or substituted and wherein R is a linear, branched, or cyclic alkyl, alkenyl, or alkynl. Examples of heterocycles with 4-6 total atoms (as an example) are oxetane, tetrahydofuran, morpholine, furan, piperazine, pyridine, thiazole, oxazole, tetrahydropyran, and thiomorpholine dioxide.

The term “substituted” means that at least one hydrogen atom (—H) bonded to a carbon atom or heteroatom of a corresponding unsubstituted compound or functional group is replaced by a substituent. Examples of such substituents are F, Cl, Br, OH, CF₃, NH, NHR^{N}, N(R^{N})₂. The term “—H” means a hydrogen or hydrogen radical that is covalently bonded to another atom. “Hydrogen” and “—H” are interchangeable, and unless clearly specified have identical meanings. R is a linear, branched, or cyclic alkyl, alkenyl, or alkynl.

The compositions disclosed herein have utility, e.g. for improving studies using florescent detection molecules. As disclosed herein, a buffering composition includes one or more buffering agents, one or more celluloses or derivatives thereof, where the celluloses or derivatives thereof are at a concentration of less than about 2 wt % of the buffering composition, and polyvinyl alcohol at less than about 2 wt % of said buffering composition. It was found that when multiple dyes, such as polymer dyes, are used on detection agents, that dye stacking led to unwanted stacking of the polymer dye molecules against each other when mixed and stored together, creating false double positive populations during analysis in fluorescence detection studies. The compositions as provided herein prevent such dye stacking and improve detectability of a target analyte.

A composition as provided herein is a buffering composition meaning that the composition includes at least one buffering agent that stabilizes pH of the system and resists changes in pH with acid or alkali is added to the buffering agent relative to a system without the buffering agent included. Choice of buffering agent can be readily handled by one of ordinary skill in the art but buffering agents that have a pKa in the range of 6-8 are desirable for studies of many biological systems. Illustrative examples of such buffering agents include but are not limited to sodium bicarbonate, sodium carbonate, potassium bicarbonate, potassium carbonate, potassium phosphate, sodium phosphate, ethylene diamine, imidazole, triethanolamine, TRIS (tris(hydroxymethyl)aminomethane), HEPES (4-(2-Hydroxyethyl)-1-piperazineethanesulfonic acid), or any combination thereof. Optionally, a buffering agent excludes HEPES. A buffering agent is optionally present in the composition at a concentration of 0.01 millimolar (mM) to 10 molar (M), or any value or range therebetween. Optionally, a buffering agent is a phosphate buffering agent, illustratively sodium and/or potassium phosphates or mixture thereof as understood in the art optionally at 10 to 100 mM. Optionally, a buffering agent is phosphate buffered saline (e.g. 137 millimolar (mM) NaCl, 2.7 mM KCl, 10 mM Na₂HPO₄, and 1.8 mM KH₂PO₄ in water, or substantial equivalents thereof) as this is understood in the art.

It was found that dye stacking could be reduced or eliminated by adding particular compounds to a buffering composition and improved to a greater extent when the particular compounds were at particular concentrations or ranges thereof. As an example, it was found that with the addition of one or more celluloses or derivatives thereof in combination with polyvinyl alcohol (PVA) wherein each of the celluloses or the PVA are present at no greater than 2 weight percent (wt %) of the buffering composition, that dye stacking such as that observed most particularly with polymer dyes is reduced or eliminated.

As such, a buffering composition as provided herein includes one or more celluloses or derivatives thereof. Optionally, a buffering composition includes 1, 2, 3, 4 or more celluloses or derivatives thereof. Optionally, a buffering composition includes 1-3 celluloses or derivatives thereof. Optionally, a buffering composition includes 1-2 celluloses or derivatives thereof. Optionally, a buffering composition includes 1 cellulose or derivative thereof.

A cellulose is optionally a linear or branched alkyl cellulose. An alkyl cellulose optionally includes from hundreds to thousands of β(1→4) linked D-glucose units wherein one or more of the glucose units are modified by the addition of an alkyl on one or more hydroxyls in the structure. Optionally, an alkyl cellulose is a linear alkyl cellulose meaning that the alkyl cellulose includes one or more linear alkyl groups optionally excluding a branched alkyl. Optionally, an alkyl cellulose is a branched alkyl cellulose meaning that the alkyl cellulose includes one or more branched alkyl groups (e.g. isobutane). Optionally, an alkyl is bound to the glucose at a carbon on the alkyl that is not a terminal carbon. Optionally, a cellulose is a cellulose derivative. A cellulose derivative is optionally substituted at one or more positions with an alkyl, alkenyl, or alkynyl as defined herein, optionally a hydroxyalkyl, a carboxyalkyl, or other such derivative.

An alkyl on an alkyl cellulose is optionally a C₁-C₄ alkyl, optionally a C₁ alkyl, optionally a C₂ alkyl, optionally linear or branched a C₃ alkyl, optionally, a linear or branched C₄ alkyl. In some aspects, a cellulose is a methyl cellulose (MC) wherein one or more hydroxyls on the glucose is substituted with a C₁ alkyl. A methyl cellulose is optionally methyl cellulose with 12-18 cP measured at 2% in H₂O at 20° C. Optionally, a methyl cellulose is available from Sigma-Aldrich (St. Louis, MO) as product number M7027.

In some aspects, a cellulose is a hydroxypropyl cellulose (HC) wherein one or more of the hydroxyls on the glucose units are substituted with a hydroxylated C₃ alkyl. Optionally, a hydroxypropyl cellulose includes one or more CH₂CH₂(OH)CH₃ on 1, 2 or 3 hydroxyl locations on the glucose unit. Optionally, hydroxypropyl cellulose includes a CH₂CH₂(OH)CH₃ on three of the hydroxyl locations on the glucose unit. Optionally, a hydroxypropyl cellulose is of CAS number 9004-64-2, and optionally has a viscosity of 3-6 mPas, 2% in water at 20° C. and is optionally sold as product number H047325G by Fisher Scientific, Waltham, MA.

A cellulose is present in a composition at an amount of greater than zero to about 2 wt %. Optionally, a cellulose is present in a composition at from about 0.5 wt % to about 1 wt %. Optionally, a cellulose is present in a composition at from about 0.75 wt % to about 1 wt %. Optionally, a cellulose is present in a composition at from about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %.

Optionally, a cellulose includes a methyl cellulose, the methyl cellulose present at about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %.

Optionally, a cellulose includes a hydroxypropyl cellulose, the hydroxypropyl cellulose present at about 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %.

A buffering composition as provided herein optionally further includes one or more alcohols. In some aspects, an alcohol is a polyvinyl alcohol (PVA). Polyvinyl alcohol is a homopolymer of ethanol and includes a predominant of 1,3-diol linkages but may also include some level of 1,2-diol linkages. PVA is optionally hydrolyzed or non-hydrolyzed. PVA of a molecular weight of ˜10,000-26,000 Da may illustratively be used. Such PVA may be as in CAS Number: 9002-89-5, 25213-24-5, or a combination thereof. PVA is optionally present in a composition at a weight percent of greater than zero wt % to 2 wt % of said buffering composition. Optionally, PVA is present in a buffering composition at 0.5 wt %, optionally about 0.55 wt %, optionally about 0.6 wt %, optionally about 0.65 wt %, optionally about 0.7 wt %, optionally about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %, optionally about 1.6 wt %, optionally about 1.7 wt %, optionally about 1.8 wt %, optionally about 1.9 wt %, optionally about 2.0 wt %.

A buffering composition as provided herein optionally includes a combination of methyl cellulose and PVA. Optionally, composition includes about 0.75 wt % to 1.5 wt % methyl cellulose and about 0.5 wt % polyvinyl alcohol. Optionally, a composition includes 0.5 wt % PVA and methyl cellulose at about 0.75 wt %, optionally about 0.8 wt %, optionally about 0.85 wt %, optionally about 0.9 wt %, optionally about 1.0 wt %, optionally about 1.1 wt %, optionally about 1.2 wt %, optionally about 1.3 wt %, optionally about 1.4 wt %, optionally about 1.5 wt %. In some aspects, a composition includes about 1 wt % methyl cellulose and about 0.5 wt % polyvinyl alcohol.

A buffering composition optionally includes a combination of hydroxypropyl cellulose and PVA. Optionally, composition includes about 0.75 wt % to 1.0 wt % hydroxypropyl cellulose and about 0.5 wt % polyvinyl alcohol. Optionally, a composition includes 0.5 wt % PVA and hydroxypropyl cellulose at about 0.75 wt %, optionally about 0.8 wt %, optionally about 0.85 wt %, optionally about 0.9 wt %, optionally about 1.0 wt %. In some aspects, a composition includes about 0.75 wt % hydroxypropyl cellulose and about 0.5 wt % polyvinyl alcohol.

It is appreciated that a buffering composition optionally further includes one or more salts as are typically included in a buffer as understood in the art, optionally NaCl, KCl, MgClh, among others, or combinations thereof. A salt is optionally present at a concentration of 0.1 mM to 250 mM, or any value or range therebetween.

Not all compounds are suitable for use in a buffering composition as provided herein. For example, certain salts such as CaCl₂ or ammonium chloride negatively affect the ability to reduce or eliminate stacking or otherwise negatively effects assay performance. Some other compounds have no effect or a negative on reducing or eliminating stacking or production of double positive populations of cells due to stacking interactions between dye molecules. For example, a buffer composition optionally excludes CaCl₂, ammonium chloride, diazolidinyl urea, dinitrobenzenesulfonate (DNBS), bis-hydroxy urea, formaldehyde, propyl gallate, saponin, bishydroxy methyl urea, casein, a dextran, polyethylene glycol, glucose (free glucose), 4-((4-(dimethylamino)phenyl)azo)benzoyl (Dabcyl), oligonucleotides (e.g. QSY® 21, ssDNA), HEPES (some aspects), tween, ethanol, heparin, phloretin, polyvinylpyrrolidone (PVP), ethylenediaminetetraacetic acid (EDTA), sulfuric acid, carrageenan, CHAPS ((3-((3-cholamidopropyl) dimethylammonio)-1-propanesulfonate), dimethyl sulfoxide (DMSO), dimethyl formamide, fetal bovine serum, or any combination thereof.

In some aspects, a buffering composition further includes one or more additives. An additive is optionally a sugar, a protein additive, or the like, or combinations thereof. Illustrative examples of an additive include a sugar, optionally fructose, sucrose, or the like. When present, a sugar may be included at about 0.1 wt % to about 5 wt % or other desirable concentration. In some aspects, a sugar is present at about 0.1 wt %, optionally about 0.25 wt %, optionally about 0.5 wt %, optionally about 0.75 wt %, optionally about 1.0 wt %, optionally about 1.25 wt %, optionally about 1.5 wt %, optionally about 1.75 wt %, optionally about 2.0 wt %, optionally about 2.25 wt %, optionally about 2.5 wt %, optionally about 2.75 wt %, optionally about 3.0 wt %, optionally about 3.25 wt %, optionally about 3.5 wt %, optionally about 3.75 wt %, optionally about 4.0 wt %, optionally about 4.25 wt %, optionally about 4.5 wt %, optionally about 4.75 wt %, optionally about 5.0 wt %.

An additive is optionally a protein additive. Illustrative examples of a protein additive include but are not limited to an albumin or the like. A protein additive is optionally an albumin, optionally bovine serum albumin or human serum albumin. A protein additive is optionally included at about 0.1 wt % to about 5 wt % or other desirable concentration. In some aspects, a protein additive is present at about 0.1 wt %, optionally about 0.25 wt %, optionally about 0.5 wt %, optionally about 0.75 wt %, optionally about 1.0 wt %, optionally about 1.25 wt %, optionally about 1.5 wt %, optionally about 1.75 wt %, optionally about 2.0 wt %, optionally about 2.25 wt %, optionally about 2.5 wt %, optionally about 2.75 wt %, optionally about 3.0 wt %, optionally about 3.25 wt %, optionally about 3.5 wt %, optionally about 3.75 wt %, optionally about 4.0 wt %, optionally about 4.25 wt %, optionally about 4.5 wt %, optionally about 4.75 wt %, optionally about 5.0 wt %.

The remainder of the buffering composition optionally includes water, optionally purified water or any water suitable for use in biological analyses such as flow cytometry and the like.

A buffering composition as used herein may be combined with one or more analytical agents optionally for the detection, characterization, purification, or observation of a target analyte in a system. An analytical agent is optionally any molecule that may be used to bind to a target, optionally selectively bind a target optionally wherein the K_{d} for the desired target is at least 10-fold lower for the target than for any other molecule naturally found in a organism. A target is any structure desired to be labeled, optionally a tissue, a cell, a protein, a nucleic acid, a lipid, or any other desired structure.

An analytical agent is any molecule that may bind a target, and optionally, selectively bind a target. An analytical agent may be a protein, nucleic acid (double or single stranded DNA, RNA, or other), hormone, neurotransmitter, sugar, organic or inorganic small molecule (e.g. 1000 Da or smaller), or other material that is able to selectively bind a target. Illustrative examples of an analytical agent include, but are not limited to an antibody, such as any suitable antibody known in the art including other immunologically active fragments of antibodies or single chain antibodies. An analytical agent may be a single moiety, e.g., a polypeptide or protein, or it may include two or more moieties, e.g., a pair of polypeptides such as a pair of single chain antibody domains. A polypeptide is optionally any molecule with 50 or fewer amino acids in a continuous chain. An antibody may be a monoclonal antibody or a polyclonal antibody. An antibody or fragment thereof may be derived from an organism such as sheep, goat, rabbit or rat.

An analytical agent may be an antibody fragment such as single chain antibodies (scFv), Fab and scFv antibodies, single domain antibodies (VHH), or chimeric antibodies. The analytical agent may be derived from a naturally occurring protein or polypeptide; it may be designed de novo, or it may be selected from a library. Optionally, an analytical agent may be derived from an antibody, a single chain antibody (scFv), a single domain antibody (VHH), a lipocalin, a single chain MHC molecule, an Anticalin™ (Pieris), an Affibody™, a nanobody (Ablynx) or a Trinectin™ (Phylos).

In some aspects, an analytical agent is a fragment of an antibody, optionally an antigen binding fragment or a variable region. Examples of antibody fragments include Fab, Fab′, F(ab′)₂ and Fv fragments. Additional analytical agent molecules include diabodies, linear antibodies, single-chain antibody molecules, and multispecific antibodies formed from antibody fragments.

An analytical agent may be linked to one or more dyes. A dye as used herein may be a fluorophore. A fluorophore may be a rhodamine dye, a fluorescein dye, a coumarin dye, an indole dye, or any combination thereof. Optionally, a dye may be or include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor®350, Alexa Fluor®430, Alexa Fluor®488, Alexa Fluor®532, Alexa Fluor®546, Alexa Fluor®568, Alexa Fluor®594, Alexa Fluor®633, Alexa Fluor®647, Alexa Fluor®660, Alexa Fluor®680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.

It was found that the buffering composition as used herein is particularly suited to the use of polymer dyes, many of which are themselves often subject to stacking interactions, which are reduced or eliminated by the use of the presence buffering composition. Illustrative examples of polymer dyes that may be used in any aspect provided herein include but are not limited to those described in U.S. Pat. No. 8,354,239, International application publication numbers WO2004/000970 or WO 2020/187919, or dyes sold as brilliant violet dyes by BD Biosciences (Franklin Lakes, NJ). In some aspects, the dye is Brilliant Violet 421™, Brilliant Violet 570™ Brilliant Violet 605™, Brilliant Violet 650™, Brilliant Violet 711™, or Brilliant Violet 785™.

As such, also provided are cocktails that can be used for analyzing a biological sample wherein the cocktail includes a buffer composition as provided herein combined with one or more analytical agents conjugated to one or more dyes. It was found that the buffer composition as provided herein is capable of reducing or eliminating dye stacking interactions thereby improving analytical results. In some aspects, a cocktail is provided in dry format whereby a buffer composition is combined with one or more dye conjugated analytical agents and subjected to drying. The process of drying in the presence of the one or more analytical agents prevents dye stacking interactions that occurs in the absence of the buffer composition as provided herein such that when a cocktail is combined with a biological sample, the analytical agent is more effectively able to specifically bind a target and dramatically reduce or eliminate false double positive populations that are produced by stacking of multiple target analytes.

A cocktail as provided herein includes a buffer composition as provided herein and one or more analytical agents bound to a dye molecule, and optionally one or more additives as provided herein. Optionally, an analytical agent is an antibody, fragment hereof, or derivative thereof, as provided herein and conjugated to one or more dye molecules, optionally brilliant violet dyes. Prior to combination with an analytical agent, after combination with an analytical agent, or both, a cocktail may be subjected to drying to remove residual water. A cocktail may be dried by any method known in the art including but not limited to vacuum drying, lyophilization, vitrification, or other such technique. The cocktail is optionally dried to a water content of 10 wt % or less, optionally 2 wt % or less, optionally 1 wt % or less water.

Also disclosed herein are methods of detecting the presence or absence of an analyte/target in a sample (optionally a biological sample) or an organism. The methods may include contacting a sample with a buffer composition or cocktail as disclosed herein, to form an analysis composition and subjecting the analysis composition to analysis by detecting one or more of the dyes in contact with a portion of the biological sample. The detecting may utilize one or more various analytical methods illustratively but not limited to flow cytometry, FISH, immunohistochemistry, sandwich assay, Southern blot, western blot, microarray, or substrate binding assay.

Optionally, a sample is a biological sample containing one or a plurality of cells. Such a biological sample is optionally a cell or tissue or plurality thereof. In some aspects, a biological sample is plasma, serum, whole blood, saliva, urine, feces, lymph, nasal secretions, or other desired sample. In some aspects, a biological sample is whole blood or plasma. Optionally, a sample is plasma. An analysis composition may be formed by contacting a biological sample to a cocktail that is dried as provided herein thereby reconstituting the analytical agent in the cocktail freeing it to bind a target within the biological sample. In other aspects, the cocktail is contacted with a biological agent forming an analysis composition followed by drying the analysis composition. Such a dried material may be reconstituted prior to analysis by contact with a second buffering composition, water or other. Following contacting of the biological sample with the cocktail, the analysis composition is subjected to analysis by detecting one or more analytical agents in contact with a portion of said biological sample.

In some aspects, a method includes forming an analysis composition by contacting a biological sample comprising plasma or whole blood with a dried down cocktail comprising the buffer composition as provided herein and one or more antibodies, said one or more antibodies conjugated to one or more florescent dyes, optionally a polymer dyes, thereby reconstituting antibodies and subjecting the analysis composition to analysis by flow cytometry.

In some aspects, the cocktail used in a method of analyzing a biological sample includes two or more antibodies specific for different target molecules, wherein an antibody specific for a first target molecule is conjugated to a first dye, and wherein an antibody specific for a second target molecule is conjugated to a second dye, where the first dye and the second dye are characterized by different fluorescent properties. Optionally, a first dye, a second dye or both are a polymer dye. Optionally, both a first dye and a second dye are polymer dyes. Optionally, a cocktail includes 2-20 different antibodies or derivatives thereof each conjugated with a different dye, optionally including at least one polymer dye, optionally a brilliant violet dye. In some aspects, at least one antibody or derivative thereof is conjugated with a polymer dye (optionally Brilliant Violet 421™, Brilliant Violet 570™, Brilliant Violet 605™, Brilliant Violet 650™ Brilliant Violet 711™, or Brilliant Violet 785™), and at least one antibody or derivative thereof is conjugated with a different dye type, optionally a rhodamine dye, a fluorescein dye, a coumarin dye, an indole dye, or any combination thereof.

Various aspects of the present disclosure are illustrated by the following non-limiting examples. The examples are for illustrative purposes and are not a limitation on any practice of the present disclosure. It will be understood that variations and modifications can be made without departing from the spirit and scope of the disclosure. Reagents illustrated herein are commercially available or readily synthesized by well-known methods from readily commercially available precursors, and a person of ordinary skill in the art readily understands where such reagents may be obtained.

### EXAMPLES

### Example 1

Buffering compositions were made using various amounts of methyl cellulose or hydroxypropyl cellulose and combined with PVA in water. 5 wt % stock solutions were prepared for each of methyl cellulose (M7027; Sigma-Aldrich), hydroxypropyl cellulose (H047325G; Fisher Scientific, Waltham, MA) and PVA (CAS 9002-89-5; 44.05 g/mol; Fisher Scientific, Waltham MA) by adding 2.5 grams (g) to 1×PBS (0.87 wt % sodium chloride, 0.14 wt % Sodium phosphate monobasic), respectively. 2.5 g of each compound was partially dissolved in 35 milliliter(s) (ml) PBS in a 50 ml conical tube and placed on a rocker for 3-4 hours. Then the tubes were transferred into a refrigerator overnight. The next day, volumes were adjusted to 50 ml with 1×PBS. A first buffer composition was made by combining the stock solutions to form a 1 wt % methyl cellulose and 0.5 wt % PVA solution. A second buffer composition was made by combining the stock solutions to form a 1 wt % hydroxypropyl cellulose and 0.5 wt % PVA solution.

Individual brilliant violet dye (BV) conjugated antibodies were marinated by incubating the antibody (0.1-0.5 μg) with the first or second buffer composition by adding the antibody to the buffer composition in appropriate concentrations in a smallest volume possible. 0.5 wt % of Bovine Serum Albumin (BSA) in PBS (1×) was used to adjust the mixture to the final volume. Antibodies tested were anti-CD19 conjugated to Brilliant Violet™ 711(BV711), anti-CD4 conjugated to Brilliant Violet™ 650 (BV650), anti-CD4 conjugated to Brilliant Violet™ 750 (BV750), anti-CD56 conjugated to Brilliant Violet™ 737 (BV737), anti-CD8 conjugated to Brilliant Violet™ 570 (BV570), anti-CD8 conjugated to Brilliant Violet™ 711 (BV711), anti-CD28 conjugated to Brilliant Violet™ 785 (BV785), anti-CD14 conjugated to Brilliant Violet™ 605 (BV605), anti-CD16 conjugated to Brilliant Violet™ 421 (BV421). Antibodies were obtained from Cytek Biosciences and conjugated by standard techniques.

Cocktails were formulated either using “marinated” BV-conjugated antibodies and “non-marinated” non-BV-conjugated antibodies or “non-marinated” BV and non-BV antibodies. In both cases, the final concentration of cellulose was 1 wt % and the concentration of PVA was 0.5 wt % in the cocktails. In addition, cocktails were also supplemented with 2.5 wt % sucrose and 1.5 wt % Bovine Serum Albumin (BSA) to facilitate the drying process. Cocktail volume was kept as low as possible. Single test volumes were dispensed into FACS tubes, to the center of the bottom of the tube.

The cocktails were subjected to drying by lyophilization using a standard 3-day dry down cycle.

Human whole blood was obtained from consenting donors and used as a biological sample for studies of the ability of a buffer composition as provided herein to reduce or eliminate the presence of false double populations using the above antibody cocktails. 10 μl of the respective buffer composition is added to each study tube followed by the addition of desired antibodies and bringing volume up to 50 μl using cell stain buffer. 100 μl whole blood was added to each tube and incubated in the dark at ambient temperature for 15 min. 2 ml of 1× lysis buffer was added and incubated in the dark at ambient temperature for 15 min. The tubes were spun at 1500×g for 5 min. The supernatant was decanted and the pellet washed 2× with wash buffer. To this about 300 μl fix was added and the sample run on the flow cytometer using single color beads for compensation.

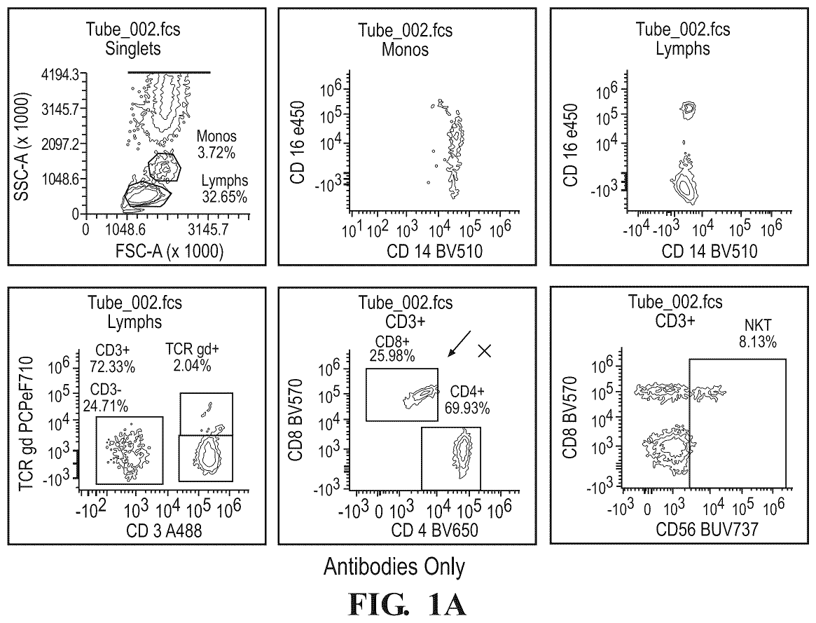

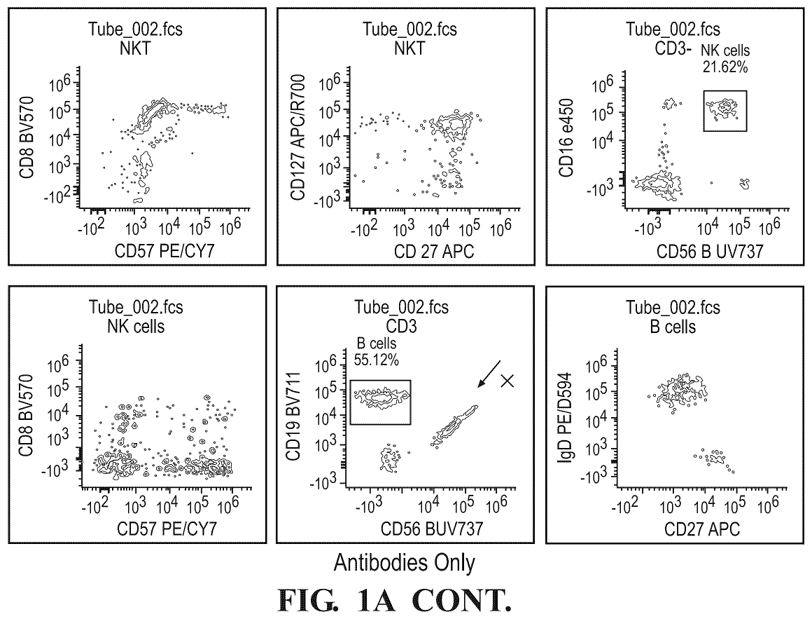

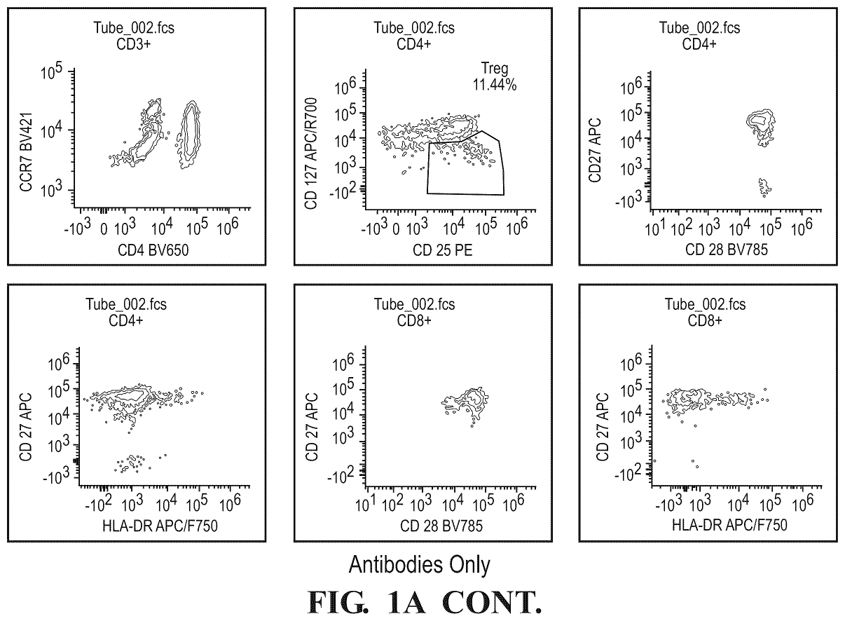

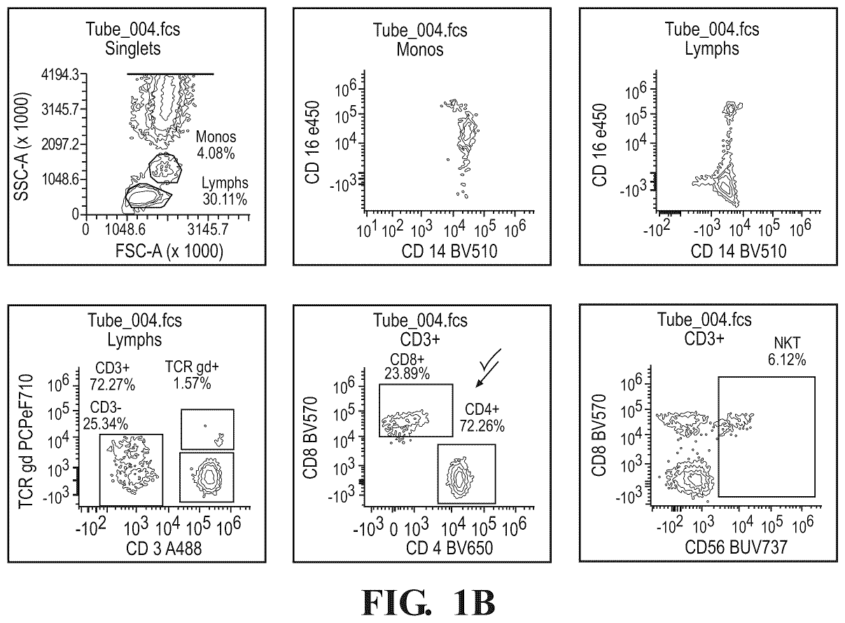

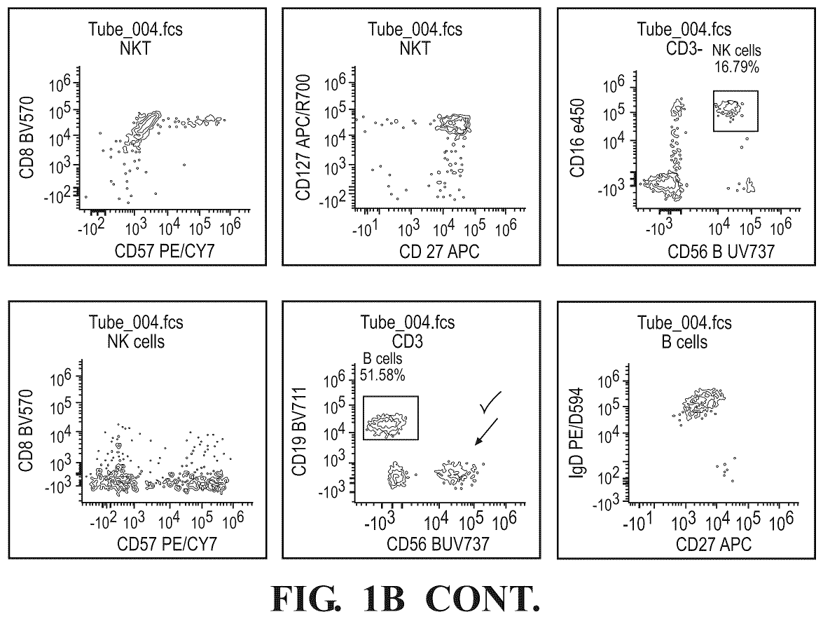

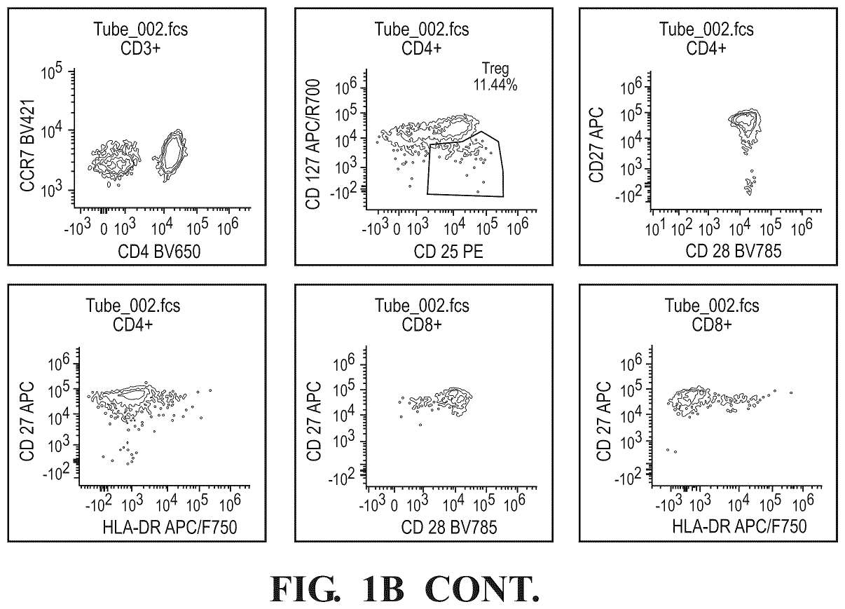

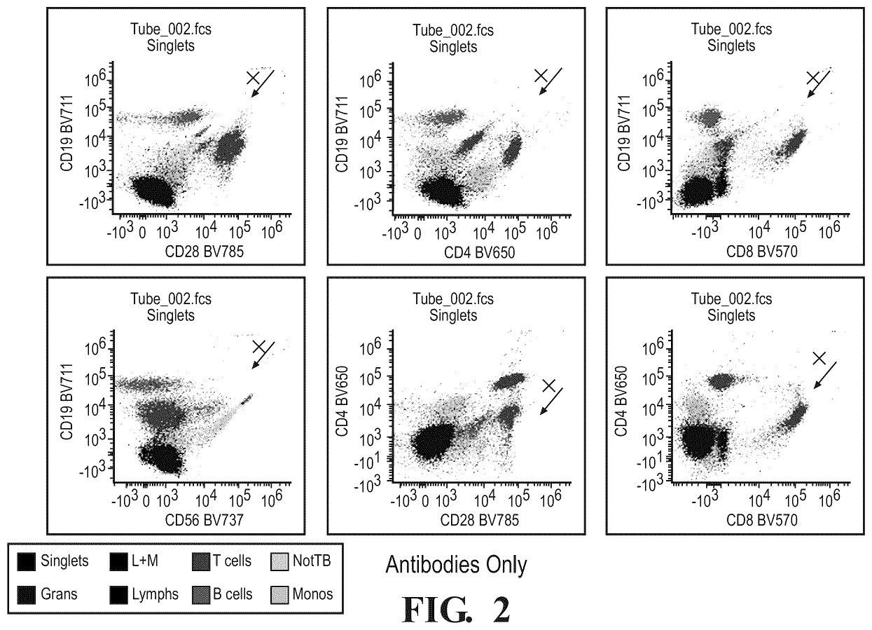

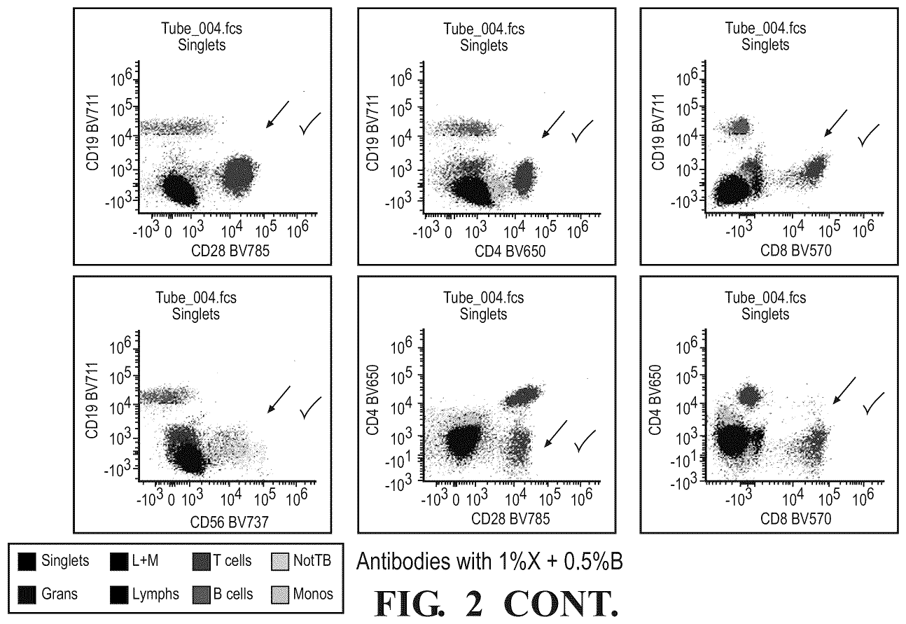

Results gating on desired populations are illustrated in FIGS. 1-5. FIG. 1A illustrates results using dye-conjugated antibodies in standard PBS buffer. While not all dye-conjugated antibodies demonstrated issues with stacking, false double positive populations were observed using CD8 and CD4 polymer dye conjugated antibodies as well as CD19 and CD56 polymer dye conjugated antibodies with the false double positive populations pointed to by arrow and the X indicating their presence. When a buffer composition of 1 wt % methyl cellulose and 0.5 wt % PVA was added to the system prior to marinating with BV-conjugated antibodies, the false double positive populations were no longer observed as illustrated in FIG. 1B with an arrow pointing to the region where false double positive populations were observed without the buffer composition and a check mark illustrating their absence.

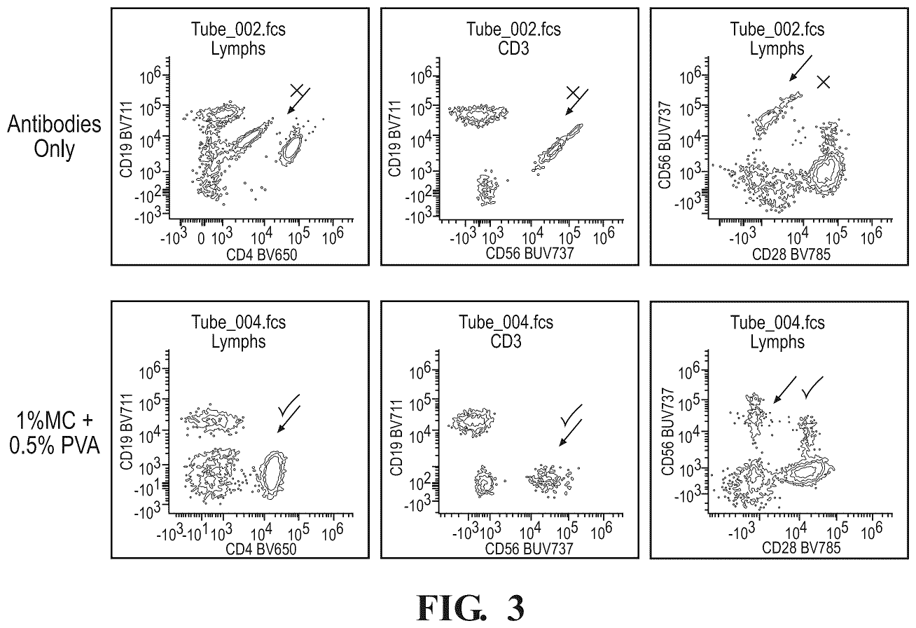

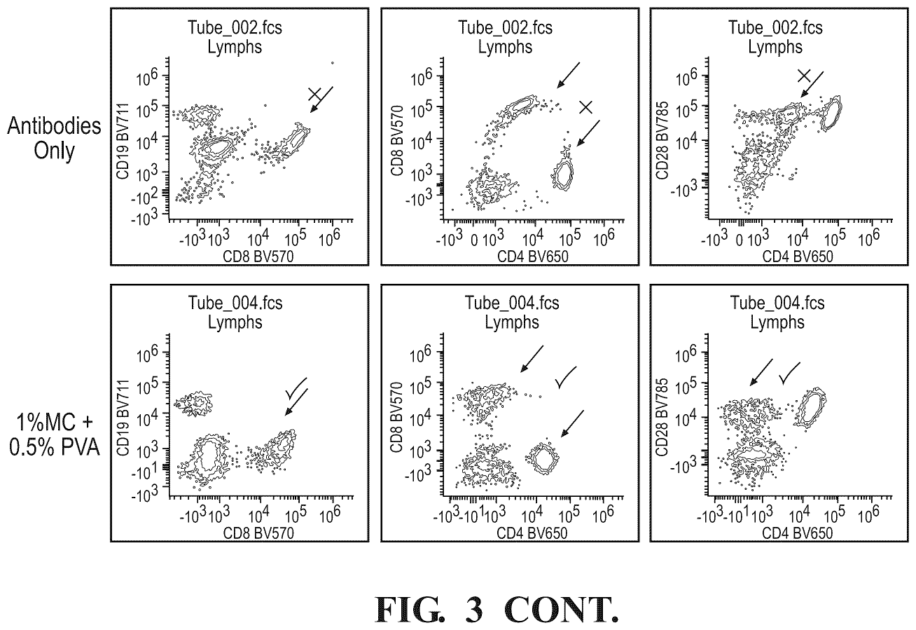

Similar studies were performed with polymer dye conjugated antibodies to CD28, CD19, CD56, CD8, CD28, and CD4 where creating a buffer cocktail including the buffer composition as provided herein removes observations of false double positive populations. Results are illustrated in FIG. 3.

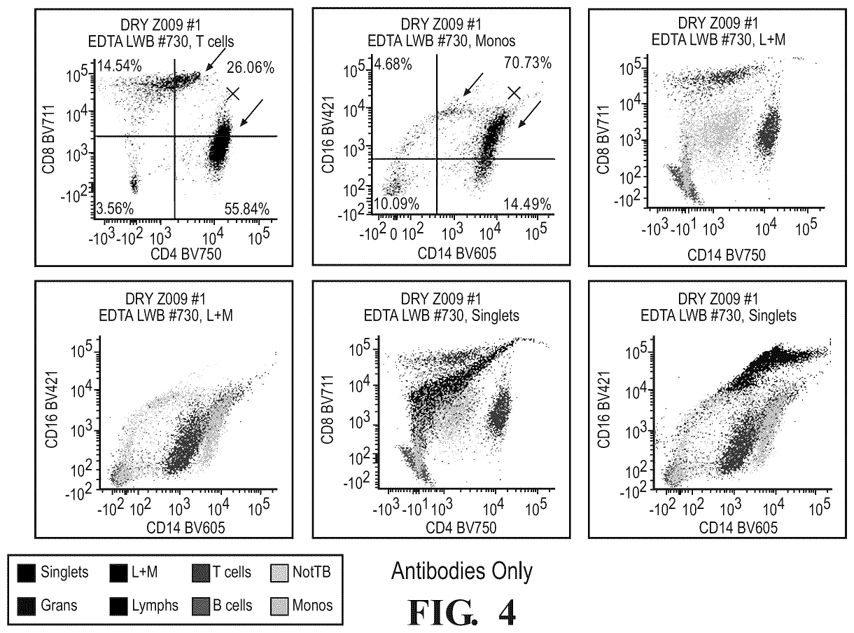

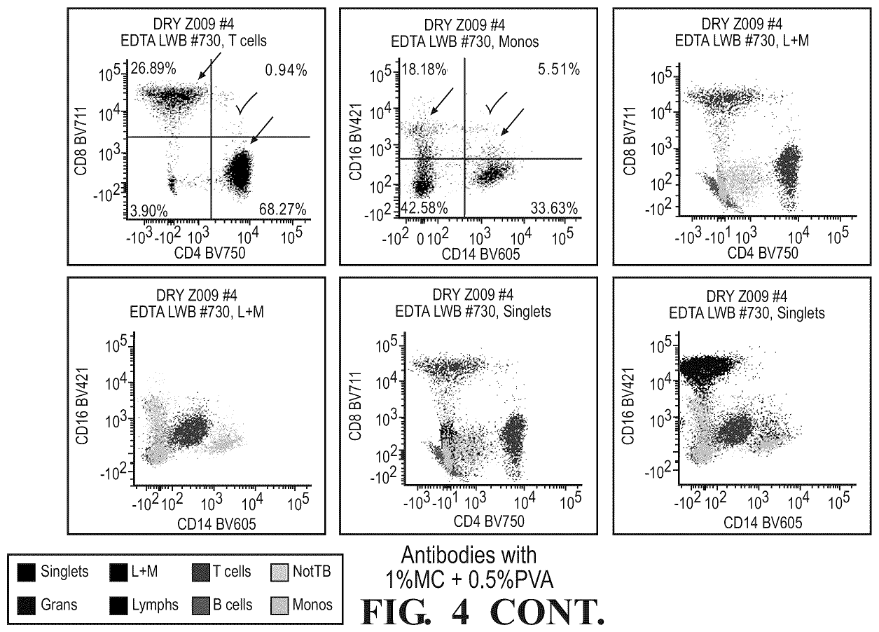

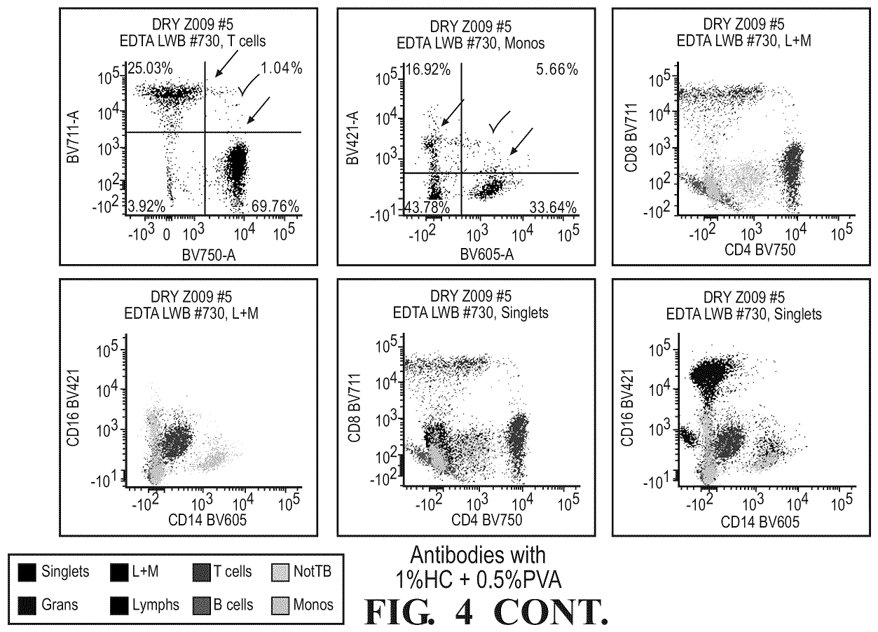

As illustrated in FIG. 4, use of polymer dye conjugated antibodies to CD28, CD19, CD56, CD8, CD28, and CD4 with standard PBS buffer generated false double positive populations of cells observed by flow cytometry. When either buffer composition of 1 wt % methyl cellulose and 0.5 wt % PVA or 1 wt % hydroxypropyl cellulose and 0.5 wt % PVA as provided herein was used observations of false double positive populations were dramatically reduced. Results are illustrated in FIG. 3.

Flow cytometry data were obtained using polymer dye conjugated antibodies to CD CD8, CD4, CD16, and CD14 whereby two buffer formulations were tested, 1 wt % methyl cellulose and 0.5 wt % PVA and 1 wt % hydroxypropyl cellulose and 0.5 wt % PVA demonstrating similar efficacy in eliminating false double positive populations in an overall seven antibody cocktail.

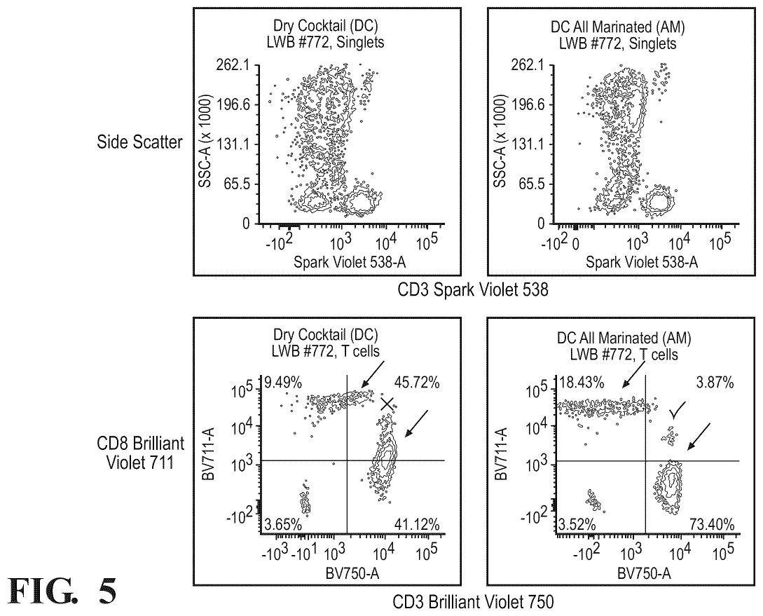

FIG. 5 illustrates flow cytometry data obtained using polymer dye conjugated antibodies to CD8 conjugated to BV711, CD3 conjugated to Spark Violet™ 538 and CD4 conjugated to BV750 and shows that incubating the antibodies with a buffer composition as provided herein reduces prevalence of false double positive populations.

### Additional Exemplary Aspects

Aspect 1. A buffering composition including:
   one or more buffering agents;
   one or more celluloses or derivatives thereof, the celluloses or derivatives thereof at a concentration of less than about 2 wt % of the buffering composition; and polyvinyl alcohol at less than about 2 wt % of the buffering composition.
Aspect 2. The buffering composition of Aspect 1, wherein the cellulose is a linear or branched alkyl cellulose.
Aspect 3. The buffering composition of any of Aspects 1 or 2, wherein the alkyl cellulose is a C₁-C₄ alkyl cellulose.
Aspect 4. The buffering composition of any of Aspect 2 or 3, wherein the alkyl cellulose is methyl cellulose.
Aspect 5. The buffering composition of Aspects 1-4, wherein the cellulose is a branched chain alkyl cellulose.
Aspect 6. The buffering composition of any of Aspects 1-5, wherein the cellulose is hydroxypropyl cellulose.
Aspect 7. The buffering composition of any of Aspect 1, wherein the cellulose is a combination of methyl cellulose and hydroxypropyl cellulose.
Aspect 8. The buffering composition of any of Aspects 1-7, wherein the cellulose is present at about 0.5 wt % to about 1 wt %.
Aspect 9. The buffering composition of any of Aspects 1-7, wherein the cellulose is present at about 0.75 wt % to about 1 wt %.
Aspect 10. The buffering composition of any of Aspects 1-9, wherein the buffering agent is characterized by a pKa of 6-8.
Aspect 11. The buffering composition of any of Aspects 1-10, wherein said buffering agent comprises a phosphate.
Aspect 12. The buffering composition of any of Aspects 1-11, wherein the buffering agent is phosphate buffered saline.
Aspect 13. The buffering composition of any of Aspects 1-12, wherein the composition further comprises an albumin, optionally bovine serum albumin or human serum albumin.
Aspect 14. The buffering composition of any of Aspects 1-13, wherein the composition includes about 0.75 wt % to 1.5 wt % methyl cellulose and about 0.5 wt % polyvinyl alcohol.
Aspect 15. The buffering composition of any of Aspects 1-13, wherein the composition includes about 1 wt % methyl cellulose or about 0.75 wt % to about 1 wt % hydroxypropyl cellulose, and about 0.5 wt % polyvinyl alcohol.
Aspect 16. The buffering composition of any of Aspects 1-13, wherein the composition includes about 1 wt % methyl cellulose or about 0.75 wt % hydroxypropyl cellulose, and further including about 0.5 wt % polyvinyl alcohol.
Aspect 17. The buffering composition of any of Aspects 14-16, wherein said buffering agent is phosphate buffered saline.
Aspect 18. The composition of any of Aspects 1-17, further including bovine serum albumin at about 0.5 wt %.
Aspect 19. A method of analyzing a biological sample comprising:
   obtaining a biological sample,
   forming an analysis composition by contacting the biological sample with a cocktail comprising the buffer composition of any one of Aspects 1-18 and one or more antibodies, the one or more antibodies conjugated to one or more florescent dyes;
   optionally drying the analysis composition; and
   subjecting the analysis composition to analysis by detecting one or more of the dyes in contact with a portion of the biological sample.
Aspect 20. The method of Aspect 19, wherein the cocktail includes two or more antibodies specific for different target molecules, wherein an antibody specific for a first target molecule is conjugated to a first dye, and wherein an antibody specific for a second target molecule is conjugated to a second dye, the first dye and said second dye characterized by different fluorescent properties.
Aspect 21. The method of any of Aspects 19-20, including 1 to 23 antibodies, optionally 3 to 23 antibodies or more directed to different target molecules and each conjugated to a different dye.
Aspect 22. The method of any of Aspects 19-21, wherein at least two of the antibodies are conjugated to different brilliant violet dyes.
Aspect 23. The method of any of Aspects 19-22, wherein the dyes include one or more polymer dyes, optionally Brilliant Violet 421™, Brilliant Violet 510™, Brilliant Violet 570™, Brilliant Violet 605™, Brilliant Violet 650™, Brilliant Violet 711™, Brilliant™ Violet 750, or Brilliant Violet 785™
Aspect 24. The method of any of Aspects 19-23, wherein one or more dyes include a rhodamine dye, a fluorescein dye, a coumarin dye, an indole dye, or any combination thereof.
Aspect 25. The method of Aspect 24, wherein said dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor®350, Alexa Fluor®430, Alexa Fluor®488, Alexa Fluor®532, Alexa Fluor®546, Alexa Fluor®568, Alexa Fluor®594, Alexa Fluor®633, Alexa Fluor®647, Alexa Fluor®660, Alexa Fluor®680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.
Aspect 26. The method of any of Aspects 19-25, including drying the analysis sample, wherein the cocktail includes methyl cellulose and polyvinyl alcohol, optionally about 1 wt % methyl cellulose and about 0.5 wt % polyvinyl alcohol.
Aspect 27. The method of any of Aspects 19-25, wherein the cocktail includes methyl cellulose or hydroxypropyl cellulose, and further including polyvinyl alcohol, optionally about 1 wt % methyl cellulose, about 0.75 wt % to 1 wt % hydroxypropyl cellulose, and about 0.5 wt % polyvinyl alcohol.
Aspect 28. The method of any of Aspects 19-25, wherein the cocktail includes about 0.75 wt % methyl cellulose or about 0.75 wt % hydroxypropyl cellulose, and further including about 0.5 wt % polyvinyl alcohol.
Aspect 29. The method of any of Aspects 19-28, wherein the subjecting is by flow cytometry, FISH, immunohistochemistry, sandwich assay, Southern blot, western blot, microarray, or substrate binding assay.
Aspect 30. The method of any of Aspects 19-28, wherein the subjecting is by flow cytometry.
Aspect 31. A method of forming an analysis composition for use in analysis of a biological sample including:
   combining an analysis composition by contacting the biological sample with a cocktail including the composition of any one of Aspects 1-18 and one or more antibodies, the one or more antibodies conjugated to one or more florescent dyes; and drying the analysis composition.
Aspect 32. The method of Aspect 31, wherein the cocktail includes methyl cellulose and polyvinyl alcohol, optionally about 1 wt % methyl cellulose and about 0.5 wt % polyvinyl alcohol.
Aspect 33. The method of any of Aspects 31-32, wherein the dyes include one or more polymer dyes, optionally Brilliant Violet 421™, Brilliant Violet 570™, Brilliant Violet 605™, Brilliant Violet 650™, Brilliant Violet 711™, or Brilliant Violet 785™ Aspect 34. The method of any of Aspects 31-32, wherein one or more dyes include a rhodamine dye, a fluorescein dye, a coumarin dye, an indole dye, or any combination thereof.
Aspect 35. The method of any of Aspects 31-34 further including reconstituting the analysis composition after said step of drying.
Aspect 36. The method of Aspect 35, wherein the reconstituting is by combining the analysis composition with a liquid biological sample

### Selected Definitions

2-[3-[3-[6-[(2,5-dioxo-1-pyrrolidinyl)oxy]-6-oxohexyl]-2(3H)-benzoxazolylidene]-1-propen-1-yl]-3-ethyl-benzoxazolium, monoiodide (Cy2), Sulfo-Cyanine3 (Cy3), Cyanine 3.5 (Cy3.5), Sulfo-Cyanine5 (Cy5), Cyanine 5.5 (Cy5.5), Sulfo-Cyanine7 (Cy7), PerCP (peridinin chlorophyll-a Protein), JOE (6-carboxy-4′,5′-dichloro-2′,7′-dimethoxyfluorescein), ROX (5-(and -6)-carboxy-X-rhodamine), 7-amino-3-[2-(2,5-dioxopyrrolidin-1-yl)oxy-2-oxoethyl]-4-methyl-2-oxochromene-6-sulfonic acid (Alexa Fluor®350), N,N-Diethylethanaminium-[9-{6-[(2,5-dioxo-1-pyrrolidinyl)oxy]-6-oxohexyl}-8,8-dimethyl-2-oxo-4-(trifluormethyl)-8,9-dihydro-2H-pyrano[3,2-g]chinolin-6-yl]methansulfonate (Alexa Fluor®430), dilithium; 3-(3-amino-6-imino-5-sulfo-4-sulfonatoxanthen-9-yl)-4-carboxybenzoate (Alexa Fluor®488), ihydrogen 5-(4-{[(2,5-dioxo-1-pyrrolidinyl)oxy]carbonyl}phenyl)-2,3,3,7,7,8-hexamethyl-2,3,7,8-tetrahydro-1H-pyrrolo[3′,2′:6,7]chromeno[3,2-f]indole-10,12-disulfonate (Alexa Fluor®532), sodium 6-(2-carboxy-3,4,6-trichloro-5-{[2-({6-[(2,5-dioxopyrrolidin-1-yl)oxy]-6-oxohexyl}amino)-2-oxoethyl]thio}phenyl)-2,2,4,8,10,10-hexamethyl-3,4,5a,8,9,10,11,12a-octahydro-2H-pyrano[3,2-g:5,6-g′]diquinolin-1-ium-12,14-disulfonate (Alexa Fluor®546), 4-(2,5-dioxopyrrolidin-1-yl)oxycarbonyl-2-[7,7,19,19-tetramethyl-9,17-bis(sulfomethyl)-2-oxa-6,20-diazapentacyclo[12.8.0.03,12.05,10.016,21]docosa-1(14),3,5,8,10,12,15,17,21-nonaen-13-yl]benzoic acid (Alexa Fluor®568), [13-[2-carboxy-4-(2,5-dioxopyrrolidin-1-yl)oxycarbonylphenyl]-6,7,7,19,19,20-hexamethyl-17-(sulfomethyl)-2-oxa-20-aza-6-azoniapentacyclo[12.8.0.03,12.05,10.016,21]docosa-1(14),3,5,8,10,12,15,17,21-nonaen-9-yl]methanesulfonate (Alexa Fluor®594), Alexa Fluor®633 found at CHEBI:137393, 3-[(2Z)-2-[(2E,4E)-5-[3,3-dimethyl-5-sulfo-1-(3-sulfopropyl)indol-1-ium-2-yl]penta-2,4-dienylidene]-3-[5-(2,5-dioxopyrrolidin-1-yl)oxy-5-oxopentyl]-3-methyl-5-sulfoindol-1-yl]propane-1-sulfonate (Alexa Fluor®647), 7-amino-4-methylcoumarin-3-acetic acid, (3-{2-[(3,5-Dimethyl-1H-pyrrol-2-yl-kappaN)methylene]-2H-pyrrol-5-yl-kappaN}propanoato)(difluoro)boron (BODIPY® FL), CAS No. 216961-93-2 (BODIPY® 530/550), 12-(2,2-difluoro-12-thiophen-2-yl-3-aza-1-azonia-2-boranuidatricyclo[7.3.0.03,7]dodeca-1(12),4,6,8,10-pentaen-4-yl)dodecanoate; hydron (BODIPY® 558/568), CAS #:150173-90-3 (BODIPY® 564/570), CAS #:150173-78-7 (BODIPY® 576/589), CAS Number: 217075-36-0 (BODIPY® 581/591), CAS #:2183512-02-7 (BODIPY® 630/650), CAS No.: 1818267-45-6 (BODIPY® 650/665), Difluoro(3-{2-[(5-phenyl-1H-pyrrol-2-yl-kappaN)methylene]-2H-pyrrol-5-yl-kappaN}propanoato)boron (BODIPY® R6G), CAS #:485397-12-4 (BODIPY® TMR), CAS #:2183473-18-7 (BODIPY® TR).

The foregoing description of particular aspect(s) is merely exemplary in nature and is in no way intended to limit the scope of the disclosure, or any related invention, its application, or uses, which may, of course, vary. The compositions or processes are described with relation to the non-limiting definitions and terminology included herein. These definitions and terminology are not designed to function as a limitation on the scope or practice of any invention disclosed herein but are presented for illustrative and descriptive purposes only. While the processes or compositions are described as an order of individual steps or using specific materials, it is appreciated that steps or materials may be interchangeable such that the description may include multiple parts or steps arranged in many ways as is readily appreciated by one of skill in the art.

Definitions as used herein are intended to supplement and illustrate, not preclude, the definitions known to those of skill in the art.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/of” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Any patents or publications mentioned in this specification are indicative of the levels of those skilled in the pertinent art. These patents and publications are herein incorporated by reference to the same extent as if each individual publication was specifically and individually indicated to be completely incorporated by reference.

One skilled in the art will readily appreciate that the present disclosure is well-adapted to carry out the objects and obtain the ends and advantages mentioned, as well as those inherent therein. The present examples along with the methods, procedures, treatments, molecules and specific compounds described herein are presently representative of specific aspects, are exemplary, and are not intended as limitations on the scope of any invention, which shall be solely defined by the foregoing claims. It will be apparent that other aspects exist and are encompassed within the spirit of any invention as defined by the scope of the claims.

1. A buffering composition comprising:
one or more buffering agents;
one or more celluloses or derivatives thereof, said celluloses or derivatives thereof at a concentration of less than about 2 wt % of said buffering composition; and
polyvinyl alcohol at less than about 2 wt % of said buffering composition.

2. The buffering composition of claim 1, wherein said cellulose is a linear or branched alkyl cellulose.

3. The buffering composition of claim 2, wherein said alkyl cellulose is a C₁-C₄ alkyl cellulose.

4. The buffering composition of claim 2, wherein said alkyl cellulose is methyl cellulose or hydroxypropyl cellulose, or a combination thereof.

5. The buffering composition of claim 1, wherein said cellulose is present at 0.5 wt % to 1 wt %.

6. The buffering composition of claim 1, wherein said buffering agent is characterized by a pKa of 6-8.

7. The buffering composition of claim 1, wherein said buffering agent comprises a phosphate.

8. The buffering composition of claim 1, wherein said composition further comprises an albumin.

9. The buffering composition of claim 1, wherein said composition comprises 0.75 wt % to 1.5 wt % methyl cellulose and 0.5 wt % polyvinyl alcohol.

10. The buffering composition of claim 1, wherein said composition comprises 1 wt % methyl cellulose or 0.75 wt % to about 1 wt % hydroxypropyl cellulose, and about 0.5 wt % polyvinyl alcohol.

11. The composition of claim 10, further comprising bovine serum albumin at about 0.5 wt %.

12. A method of analyzing a biological sample comprising:
obtaining a biological sample;
forming an analysis composition by contacting said biological sample with a cocktail comprising the buffering composition of claim 1 and one or more analytical agents, said one or more analytical agents conjugated to one or more florescent dyes;
optionally drying said analysis composition; and
subjecting said analysis composition to analysis by detecting one or more of said dyes in contact with a portion of said biological sample.

13. The method of claim 12, wherein said cocktail comprises two or more antibodies specific for different target molecules, wherein a first antibody specific for a first target molecule is conjugated to a first dye, and wherein a second antibody specific for a second target molecule is conjugated to a second dye, said first dye and said second dye characterized by different fluorescent properties.

14. The method of claim 12, comprising 3 to 23 antibodies each directed to a different target molecules and each conjugated to a different dye.

15. The method of claim 12, wherein said first antibody and said second antibody are each independently conjugated to a different brilliant violet dye.

16. The method of claim 12, wherein said dye is fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), allophycocyanin, PerCP-Cy5.5, JOE (6-carboxy-4′,5′-dichloro-2′,7′-dimethoxyfluorescein), NED, ROX (5-(and -6)-carboxy-X-rhodamine), HEX, Lucifer Yellow, Marina Blue, Oregon Green 488, Oregon Green 500, Oregon Green 514, Alexa Fluor®350, Alexa Fluor®430, Alexa Fluor®488, Alexa Fluor®532, Alexa Fluor®546, Alexa Fluor®568, Alexa Fluor®594, Alexa Fluor®633, Alexa Fluor®647, Alexa Fluor®660, Alexa Fluor®680, 7-amino-4-methylcoumarin-3-acetic acid, BODIPY® FL, BODIPY® FL-Br2, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, SPK dye, cf514, DY405, DY396XL, cf570, cf405, Spark UV™ 387, Spark Violet™ 423, Spark Violet™ 500, Spark Violet™ 538, Spark Blue™ 515, Spark Blue™ 550, Spark Blue™ 574, Spark YG™ 570, Spark YG™ 581, Spark YG™ 593, Spark NIR™ 685, Spark Red™ 718, conjugates thereof, or combinations thereof.

17. The method of claim 12, further comprising drying said analysis sample, wherein said cocktail comprises methyl cellulose and polyvinyl alcohol.

18. The method of claim 12, wherein said cocktail comprises methyl cellulose or hydroxypropyl cellulose, and further comprising polyvinyl alcohol.

19. The method of claim 12, wherein said subjecting is by flow cytometry, FISH, immunohistochemistry, sandwich assay, Southern blot, western blot, microarray, or substrate binding assay.

20. A method of forming an analysis composition for use in analysis of a biological sample comprising:
forming a cocktail comprising the buffering composition of claim 1 and one or more antibodies, said one or more antibodies conjugated to one or more florescent dyes; and
drying said analysis composition.
